(19) United States
(12) Patent Application Publication
(10) Pub. No.: US 2007/0066593 A1
(43) Pub. Date: Mar. 22, 2007

(54) Use of carbamazepine derivatives for the treatment of agitation in dementia patients

(76) Inventors: Joshua Shua-Haim, Eatontown, NJ (US); Ferenc Martenyl, Budapest (HU)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/550,380

(22) PCT Filed: Mar. 31, 2004
(86) PCT No.: PCT/EP04/03418

§ 371 Date: Jul. 12, 2006

Related U.S. Application Data
(60) Provisional application No. 60/459,338, filed on Apr. 1, 2003.

(51) Int. Cl. A61K 31/55

(52) U.S. Cl. 514/217

(57) ABSTRACT

The present invention relates to the use of a compound of formula (I) wherein (a) R₁ and R₂ together form an oxy group or (b) R₁ is hydrogen and R₂ is hydroxy or acetoxy, or a pharmaceutically acceptable salt thereof, for the manufacture of a pharmaceutical composition for the treatment of agitation, in particular behavioral agitation, especially in dementia patients; a method for the treatment of agitation; pharmaceutical compositions comprising a compound of formula I; a combination comprising (a) a compound of formula I and (b) at least one compound selected from the group consisting of nootropic plant extracts, calcium antagonists, cholinesterase inhibitors, dihydroergotoxin, nicergoline, piracetame, purine derivates, pyritinol, vincamine and vinpocetine; and a commercial package including such combination.

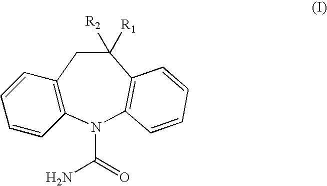

The present invention relates to new pharmaceutical uses of the carboxamides of formula I (see below). The term “carboxamides” as used herein includes, but is not limited to oxcarbazepine, 10-hydroxy-10,11-dihydrocarbamazepine and 10-acetoxy-10,11-dihydrocarbamazepine.

In particular, the invention relates to the carbamazepine derivatives of formula I 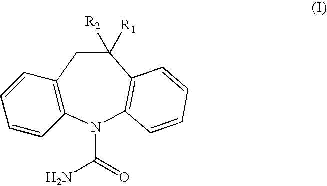
wherein (a) R₁ and R₂ together form an oxy group or (b) R₁ is hydrogen and R₂ is hydroxy or acetoxy, and to the pharmaceutically acceptable salts thereof.

If R₁ is hydrogen and R₂ is hydroxy or acetoxy, the compounds of formula I exist in two different enantiomers. The present invention relates to all such possible enantiomers.

The compound of formula I wherein R₁ and R₂ together form an oxy group is known as oxcarbazepine (10-oxo-10,11-dihydro-5H-dibenz(b,f)azepine-5-carboxamide). The preparation of oxcarbazepine and its pharmaceutically acceptable salts is described, e.g., in the German patent 2,011,087. Oxcarbazepine is marketed under the brand TRILEPTAL™. It is a known anticonvulsant drug useful in the treatment of seizures of, for example, epileptic origin.

The preparation of the compound of formula I wherein R₁ is hydrogen and R₂ is hydroxy and its pharmaceutically acceptable salts is described, e.g., in U.S. Pat. No. 3,637,661. The preparation of the compound of formula I wherein R₁ is hydrogen and R₂ is acetoxy and its pharmaceutically acceptable salts is described, e.g., in U.S. Pat. No. 5,753,646. Both compounds are described to be efficacious against epilepsy.

In accordance with the present invention, it has now surprisingly been found that the compound of formula I and the pharmaceutically acceptable salts thereof are also useful for the treatment of agitation, in particular behavioral agitation, in dementia patients, especially in patients with Alzheimer's disease.

Agitation is commonly associated with dementia and contributes to diminished quality of life for patients as well as their caregivers.

The term “dementia” as used herein relates to a condition which can be characterized as a loss, usually progressive, of cognitive and intellectual functions, without impairment of perception or consciousness caused by a variety of disorders including severe infections and toxins, but most commonly associated with structural brain disease. Characterized by disorientation, impaired memory, judgment and intellect and a shallow labile affect. The term “dementia” includes, but is not restricted to AIDS dementia, Alzheimer dementia, presenile dementia, senile dementia, catatonic dementia, dialysis dementia (dialysis encephalopathy syndrome), epileptic dementia, hebephrenic dementia, Lewy body dementia (diffuse Lewy body disease), multi-infarct dementia (vascular dementia), paralytic dementia, posttraumatic dementia, dementia praecox, primary dementia, toxic dementia and vascular dementia.

In an 8-week, open-label, single-arm study are recruited twenty-six patients community-dwelling elderly patients (>63 years of age) diagnosed with Alzheimer's disease. After a 1-week screening period to assess each patient's agitation level, oxcarbazepine 150 mg/day treatment is initiated and titrated by 150 mg/day every 3-7 days to a maximum of 1200 mg/day with a mean oxcarbazepine daily dose of 340 mg/day (range 150-552 mg/day). The primary outcome measure is improvement in behavioral agitation, based on the 38-item Cohen-Mansfield Agitation Inventory (CMAI) that evaluates the prevalence of pathologic and disruptive agitated behavior. Secondary evaluations includes effects on neuropsychiatric symptoms, cognitive impairment, global functioning, and tolerability.

Significant reductions in CMAI score from baseline (mean±SD: 60.6±14.7) occur at each timepoint (mean±SD change from baseline on Day 14: −16.9±19.7, Day 28: −22.6±19.0, Day 56: −20.8±19.6; all p≦0.0002). Significant improvement in mean Neuropsychiatric Inventory score also occur at each timepoint (all p≦0.02 versus screening). Cognitive performance and global functioning remain stable throughout the study.

As shown by this open-label, prospective study, compounds of formula I, especially oxcarbazepine, are effective and generally well tolerated in the treatment of agitation, in particular in patients with Alzheimer's disease.

For the treatment of the conditions mentioned herein, appropriate dosage will of course vary depending upon, for example, the host, the mode of administration, the specific compound or enantiomer used and the nature and severity of the condition being treated. In larger mammals, for example humans, an indicated daily dosage is in the range from about 50 to about 2000 mg, preferably from about 100 to about 1200 mg, more preferably from about 150 to about 600 mg, e.g. 340 mg, of a compound according to the invention conveniently administered, for example, in divided doses up to four times a day.

The compounds may be administered in any usual manner, e.g. orally, for example in the form of tablets or capsules, or parenterally, for example in the form of injection solutions or suspensions.

The present invention also provides pharmaceutical compositions comprising the compounds in association with at last one pharmaceutical carrier or diluent for use in the treatment of agitation. Such compositions may be manufactured in conventional manner.

Unit dosage forms may contain for example from about 2.5 mg to about 1000 mg of the compound, preferably about 300 or 600 mg.

Oxcarbazepine can be administered, e.g., in the form as it is marketed, e.g. under the trademark TRILEPTAL™.

The invention further provides the use of a compound of formula I for the manufacture of a pharmaceutical composition for the treatment of agitation.

The invention further provides a method for treatment of agitation in a subject in need of such treatment, which comprises administering to said subject a therapeutically effective amount of a compound according of formula I.

For the treatment of agitation a compound of formula I can be administered alone or in combination with a nootropic agent.

The term “nootropic agent” as used herein includes, but is not limited to nootropic plant extracts, calcium antagonists, cholinesterase inhibitors, dihydroergotoxin, nicergoline, piracetame, purine derivates, pyritinol, vincamine and vinpocetine.

The term “nootropic plant extracts” as used herein includes, but is not limited to extracts from Ginkgo leafs. The term “calcium antagonists” as used herein includes, but is not limited to cinnarizine and nimodipine. The term “cholinesterase inhibitors” as used herein includes, but is not limited to donepezil hydrochloride, rivastigmine and galantamine hydrobromide. The term “purine derivates” as used herein includes, but is not limited to pentifyllin.

Extracts from Ginkgo leafs can be administered, e.g., in the form as marketed, e.g. under the trademark Ginkodilat™ according to the information provided by the package insert. Cinnarizine can be administered, e.g., in the form as marketed, e.g. under the trademark Cinnarizin forte-ratiopharm™. Nimodipine can be administered, e.g., in the form as marketed, e.g. under the trademark Nimotop™. Donepezil hydrochloride can be administered, e.g., in the form as marketed, e.g. under the trademark Aricept™. Rivastigmine can be prepared as disclosed in U.S. Pat. No. 5,602,176. It can be administered, e.g., in the form as marketed, e.g. under the trademark Exelon™. Galantamine hydrobromide can be administered, e.g., in the form as marketed, e.g. under the trademark Reminyl™. Dihydroergotoxin can be administered, e.g., in the form as marketed, e.g. under the trademark Hydergin™. Nicergoline can be administered, e.g., in the form as marketed, e.g. under the trademark Sermion™. Piracetam can be administered, e.g., in the form as marketed, e.g. under the trademark Cerebroforte™. Pentifyllin can be administered, e.g., in the form as marketed, e.g. under the trademark Cosaldon™. Pyritinol can be administered, e.g., in the form as marketed, e.g. under the trademark Encephabol™. Vinpocetin can be administered, e.g., in the form as marketed, e.g. under the trademark Cavinton∩.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

Hence, the present invention pertains also to a combination comprising a compound of the invention, and at least one compound selected from the group consisting of nootropic plant extracts, calcium antagonists, cholinesterase inhibitors, dihydroergotoxin, nicergoline, piracetame, purine derivates, pyritinol, vincamine and vinpocetine, in which the active ingredients are present in each case in free form or in the form of a pharmaceutically acceptable salt and optionally at least one pharmaceutically acceptable carrier, for simultaneous, separate or sequential use, especially for use in a method of treating agitation.

Such a combination can be a combined preparation or a pharmaceutical composition.

The term “a combined preparation”, as used herein defines especially a “kit of parts” in the sense that the active ingredients as defined above can be dosed independently or by use of different fixed combinations with distinguished amounts of the ingredients, i.e., simultaneously or at different time points. The parts of the kit can then, e.g., be administered simultaneously or chronologically staggered, that is at different time points and with equal or different time intervals for any part of the kit of parts. Preferably, the time intervals are chosen such that the effect on the treated disease in the combined use of the parts is larger than the effect which would be obtained by use of only any one of the active ingredients.

Hence, the present invention also provides
the use of a combination as disclosed herein for the preparation of a medicament for the treatment of agitation, in particular behavioral agitation, in dementia patients, especially in patients with Alzheimer's disease; and
a commercial package comprising a combination as disclosed herein together with instructions for simultaneous, separate or sequential use thereof in the treatment of agitation, in particular behavioral agitation, in dementia patients, especially in patients with Alzheimer's disease.

In one preferred embodiment of the invention, the combination partner (b) is a cholinesterase inhibitor, e.g. rivastigmine.

Unless mentioned otherwise herein, the following dosages of the combination partners (b) can be administered to the patient:

Cinnarizine may be administered to a patient in a total daily dosage of between about 75 to about 150 mg.

Nimodipine may be administered to a patient in a total daily dosage of between about 60 to about 120 mg.

Donepezil hydrochloride may be administered to a patient in a total daily dosage of between about 5 mg and 10 mg.

Rivastigmine may be administered to a patient in a total daily dosage of between about 6 and about 12 mg.

Galantamine may be administered to a patient in a total daily dosage of between about 12 and 24 mg, e.g. 12 mg twice daily.

Dihydroergotoxin may be administered in the form of its methanesulfonate to a patient in a total daily dosage of between about 4 mg and 10 mg, e.g. about 8 mg.

Nicergoline may be administered in the form of its tartrate by intramuscular injection to a patient in a total daily dosage of between about 4 mg and 8 mg.

Piracetam may be administered to a patient in a total daily dosage of between about 1200 and 5000 mg, e.g. 4800 mg/day.

Pentifyllin may be administered to a patient in a total daily dosage of between about 400 and 800 mg.

Pyritinol may be administered in the form of its hydrochloride to a patient in a total daily dosage of about 600 mg.

Vinpocetin may be administered to a patient in a total daily dosage of between about 10 and 15 mg.

1-4. (canceled)

5. A method for the treatment of a disease selected from the group consisting of agitation, behavioral agitation and Alzheimer's disease in a subject in need of such treatment, which comprises administering to said subject a therapeutically effective amount of a compound of formula I 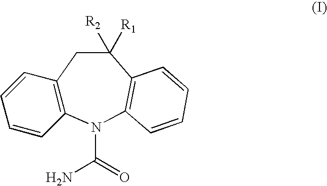
wherein (a) R₁ and R₂ together form an oxy group or (b) R₁ is hydrogen and R₂ is hydroxy or acetoxy,
or a pharmaceutically acceptable salt thereof.

6. The method according to claim 5, wherein the compound of formula I is oxcarbazepine.

7. The method according to claim 5, wherein the disease is behavioral agitation.

8. The method according to claim 5, wherein the subject to be treated is diagnosed to have Alzheimer's disease.

9. A combination comprising (a) a compound of formula I 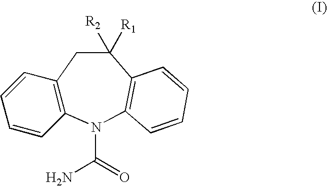
wherein (a) R₁ and R₂ together form an oxy group or (b) R₁ is hydrogen and R₂ is hydroxy or acetoxy,
and (b) at least one compound selected from the group consisting of nootropic plant extracts, calcium antagonists, cholinesterase inhibitors, dihydroergotoxin, nicergoline, piracetame, purine derivates, pyritinol, vincamine and vinpocetine, in which the active ingredients are present in each case in free form or in the form of a pharmaceutically acceptable salt and optionally at least one pharmaceutically acceptable carrier.

10. Combination according to claim 9, wherein the compound (b) is a cholinesterase inhibitor.

11. (canceled)

12. A commercial package comprising a combination according to claim 9 together with instructions for simultaneous, separate or sequential use thereof in the treatment of agitation in dementia patients.
